(19) United States
(12) Patent Application Publication
(10) Pub. No.: US 2013/0310253 A1
(43) Pub. Date: Nov. 21, 2013

(54) CHOLESTEROL LOWERING AGENT, NEUTRAL FAT LOWERING AGENT, BLOOD GLUCOSE LEVEL LOWERING AGENT, CHOLESTEROL ADSORBENT, ADSORBENT, NEUTRAL FAT ADSORBENT, HEALTHY FOOD, HEALTH SUPPLEMENT, FOOD WITH NUTRIENT FUNCTION CLAIMS, FOOD FOR SPECIFIED HEALTH USE, QUASI-DRUG, AND PHARMACEUTICAL DRUG

(71) Applicants: Seiichiro Tabata, Kanagawa (JP); Machiko Minatoya, Tokyo (JP); Hironori Iida, Kanagawa (JP); Takeshi Horie, Kanagawa (JP); Shinichiro Yamada, Kanagawa (JP); Shun Yamanoi, Kanagawa (JP)

(72) Inventors: Seiichiro Tabata, Kanagawa (JP); Machiko Minatoya, Tokyo (JP); Hironori Iida, Kanagawa (JP); Takeshi Horie, Kanagawa (JP); Shinichiro Yamada, Kanagawa (JP); Shun Yamanoi, Kanagawa (JP)

(73) Assignee: Sony Corporation, Tokyo (JP)

(21) Appl. No.: 13/983,330

(22) PCT Filed: Jan. 20, 2012
(86) PCT No.: PCT/JP2012/000341

§ 371 Date: Aug. 2, 2013

(30) Foreign Application Priority Data
Feb. 10, 2011 (JP) 2011-026859

(51) Int. Cl. A61K 33/44; A61K 9/00; A23L 1/304; A61K 31/13

(52) U.S. Cl. CPC A61K 33/44; A61K 31/13; A61K 9/0053; A23L 1/304
USPC 502/416

(57) ABSTRACT

[Object] To provide a cholesterol lowering agent, a neutral fat lowering agent, a blood glucose level lowering agent, a cholesterol adsorbent, and a neutral fat adsorbent, which have high safety.

[Solving Means] A cholesterol lowering agent, a neutral fat lowering agent, a blood glucose level lowering agent, a cholesterol adsorbent, and a neutral fat adsorbent include a porous carbon material having a specific surface area value of 10 m²/g or more and a pore volume of 0.1 cm³/g or more, the specific surface area value being measured by a nitrogen BET method, the pore volume being measured by a BJH method and an MP method.

### TECHNICAL FIELD

The present invention relates to a cholesterol lowering agent, a neutral fat lowering agent, a blood glucose level lowering agent, a cholesterol adsorbent, an adsorbent, a neutral fat adsorbent, a healthy food, a health supplement, a food with nutrient function claims, a food for specified health use, a quasi-drug, and a pharmaceutical drug.

### BACKGROUND ART

Most cholesterol in the body is catabolized in the liver to bile acids. The bile acids biosynthesized from cholesterol are secreted through the biliary tract into the intestinal tract, and help with fat absorption in the small intestine. Because 95% or more of the secreted bile acids are reabsorbed from the intestinal tract, only 5% or less of the secreted bile acids are eliminated from the body. In view of the above, as means for lowering the cholesterol level in the blood, a method of interrupting the circulation of the bile acids by the adsorption treatment of the bile acids in the intestinal tract to facilitate the catabolism of cholesterol to bile acids has been known. Then, the technique to lower cholesterol using activated carbon is well known from, for example, Japanese Patent Application Laid-open No. Sho 62-112565 and Japanese Patent Application Laid-open No. 2002-020297.

### CITATION LIST

### Patent Document

Patent Document 1: Japanese Patent Application Laid-open No. Sho62-112565
Patent Document 2: Japanese Patent Application Laid-open No. 2002-020297

### DISCLOSURE OF THE INVENTION

### Problem to be Solved by the Invention

As an agent for facilitating the catabolism of cholesterol to bile acids, cholestyramine being negative ion exchange resin is widely applied in a clinical setting. However, the daily dose of cholestyramine is large, i.e., 8 g to 12 g, and bloating sensation, constipation, or the like occurs as a side effect. Moreover, although a statin drug (HMG-CoA reductase inhibitor in the liver) has been used, the safety is compromised due to the serious side effect in some cases. For that reason, an alternative substance having an excellent cholesterol adsorption capacity with a few side effects is desired. The existing technique using activated carbon to lower cholesterol has a problem that the adsorbed amount of cholesterol is low as compared to cholestyramine. Moreover, the technique to use activated carbon to lower neutral fat or blood glucose level has not been known according to the knowledge of the present inventors.

Therefore, it is an object of the present invention to provide a cholesterol lowering agent, a neutral fat lowering agent, a blood glucose level lowering agent, a cholesterol adsorbent, a neutral fat adsorbent, and an adsorbent, which have high safety, and to provide a healthy food, a health supplement, a food with nutrient function claims, a food for specified health use, a quasi-drug, and a pharmaceutical drug, which include such a cholesterol adsorbent and/or a neutral fat adsorbent.

### Means for Solving the Problem

In order to achieve the above-mentioned object, a cholesterol lowering agent according to the present invention includes a porous carbon material having a specific surface area value of 10 m²/g or more and a pore volume of 0.1 cm³/g or more, the specific surface area value being measured by a nitrogen BET method, the pore volume being measured by a BJH method and an MP method.

The cholesterol lowering agent according to the present invention may take a form in which a cholesterol lowering capacity is not affected by coexistent protein, or a form in which the cholesterol lowering capacity is not affected by coexistent sodium chloride (electrolyte). Here, the cholesterol lowering capacity not affected by coexistent protein means that the cholesterol lowering capacity of the cholesterol lowering agent according to the present invention is not decreased even in the case where the cholesterol lowering agent according to the present invention coexists with protein, and that, from a quantitative perspective, the p-value on the cholesterol level in the blood in t-test or U-test is 0.05 or less with respect to the control not receiving the cholesterol lowering agent according to the present invention. The cholesterol lowering capacity not affected by coexistent sodium chloride (electrolyte) means that the cholesterol lowering capacity of the cholesterol lowering agent according to the present invention is not decreased even in the case where the cholesterol lowering agent according to the present invention coexists with sodium chloride (electrolyte), and that, from a quantitative perspective, the p-value on the cholesterol level in the blood in t-test or U-test is 0.05 or less with respect to the control not receiving the cholesterol lowering agent according to the present invention.

In order to achieve the above-mentioned object, a neutral fat lowering agent according to the present invention includes a porous carbon material having a specific surface area value of 10 m²/g or more and a pore volume of 0.1 cm³/g or more, the specific surface area value being measured by a nitrogen BET method, the pore volume being measured by a BJH method and an MP method.

In order to achieve the above-mentioned object, a blood glucose level lowering agent according to the present invention includes a porous carbon material having a specific surface area value of 10 m²/g or more and a pore volume of 0.1 cm³/g or more, the specific surface area value being measured by a nitrogen BET method, the pore volume being measured by a BJH method and an MP method.

In order to achieve the above-mentioned object, a cholesterol adsorbent includes according to the present invention includes a porous carbon material having a specific surface area value of 10 m²/g or more and a pore volume of 0.1 cm³/g or more, the specific surface area value being measured by a nitrogen BET method, the pore volume being measured by a BJH method and an MP method. It should be noted that the porous carbon material may be subject to a chemical treatment or molecular modification.

In order to achieve the above-mentioned object, a neutral fat adsorbent according to the present invention includes a porous carbon material having a specific surface area value of 10 m²/g or more and a pore volume of 0.1 cm³/g or more, the specific surface area value being measured by a nitrogen BET method, the pore volume being measured by a BJH method and an MP method. It should be noted that the porous carbon material may be subject to a chemical treatment or molecular modification.

The cholesterol lowering agent according to the present invention may include the neutral fat lowering agent according to the present invention, may include the blood glucose level lowering agent according to the present invention, or may include the neutral fat lowering agent according to the present invention and the blood glucose level lowering agent according to the present invention. The neutral fat lowering agent according to the present invention may include the blood glucose level lowering agent according to the present invention. The cholesterol adsorbent according to the present invention may include the neutral fat adsorbent according to the present invention.

In order to achieve the above-mentioned object, an adsorbent according to a first embodiment of the present invention includes a porous carbon material having an adsorbed amount of sodium cholate of 150 mg/g or more, and a selective adsorption rate of sodium cholate of 3 or more under existence of a molecule having a molecular weight of 1×10⁴ or more. It should be noted that examples of the molecule having a molecular weight of 1×10⁴ or more include an enzyme such as α-amylase, and protein.

In order to achieve the above-mentioned object, an adsorbent according to a second embodiment of the present invention includes a porous carbon material having an adsorbed amount of sodium cholate of 150 mg/g or more, and a selective adsorption rate of sodium cholate of 1.1 or more under existence of a molecule having a molecular weight of 1×10³ or less. It should be noted that examples of the molecule having a molecular weight of 1×10³ or less include an organic compound such as vitamin K1.

In order to achieve the above-mentioned object, an adsorbent according to a third embodiment of the present invention includes a porous carbon material having an adsorbed amount of sodium cholate of 150 mg/g or more, and a selective adsorption rate of sodium cholate of 6 or more under existence of a mineral. It should be noted that examples of the mineral include a calcium compound (specifically, for example, calcium sulfate, calcium carbide, calcium carbonate, calcium chloride, calcium oxide, calcium hydroxide, calcium phosphate, calcium phosphide, and calcium acetate) or a magnesium compound (specifically, for example, magnesium oxide, magnesium hydroxide, magnesium sulfate, magnesium carbonate, magnesium hydride, magnesium diboride, magnesium sulfide, magnesium nitride, and magnesium chloride).

Here, the selective adsorption rate of sodium cholate is defined by (adsorption rate of sodium cholate)/(adsorption rate of a substance for comparison). It should be noted that the substance for comparison is the molecule having a molecular weight of 1×10³ or less (adsorbent according the first embodiment of the present invention), molecule having a molecular weight of 1×10³ or less (adsorbent according to the second embodiment of the present invention), and mineral (adsorbent according to the third embodiment of the present invention).

In order to achieve the above-mentioned object, a healthy food according to the present invention, a health supplement according to the present invention, a food with nutrient function claims according to the present invention, a food for specified health use according to the present invention, a quasi-drug according to the present invention, or a pharmaceutical drug according to the present invention includes the cholesterol adsorbent according to the present invention including the above-mentioned favorable form and/or the neutral fat adsorbent according to the present invention including the above-mentioned favorable form. Here, the healthy food and health supplement according to the present invention are specified by the food sanitation law, the food with nutrient function claims and food for specified health use according to the present invention are specified by the health-promotion law and the food sanitation law, and the quasi-drug and pharmaceutical drug according to the present invention are specified by the pharmaceutical affairs law. Specifically, the healthy food according to the present invention means a food having a function to promote the maintenance and promotion of health, the health supplement according to the present invention means a food for supplementing the nutrients that cannot be sufficiently received from only daily meals, the food with nutrient function claims according to the present invention means a food used for supplementing nutrients (vitamin and mineral) and labeled with the function of the nutrients, the food for specified health use according to the present invention means a food labeled with an indication that the purpose of the specified health is expected to be achieved by consuming this food for those who consume this food for the purpose of the specified health in eating habits, the quasi-drug according to the present invention means one that is defined by the pharmaceutical affairs law, is categorized between a pharmaceutical drug and a cosmetic, has a mild action on the human body, and is not equipment, and the pharmaceutical drug according to the present invention means one for diagnosis, treatment, and prevention of diseases in human beings or animals by taking (internal use), applying (external use), or injecting this drug.

### Effect of the Invention

In the cholesterol lowering agent, neutral fat lowering agent, blood glucose level lowering agent, cholesterol adsorbent, neutral fat adsorbent, adsorbent, healthy food, health supplement, food with nutrient function claims, food for specified health use, quasi-drug, or pharmaceutical drug according to the present invention, because the values of the specific surface area and various pore volumes of the porous carbon material included in them (hereinafter referred to as “porous carbon material in the present invention” in some cases) are specified, it is possible to lower the cholesterol level, neutral fat level, and blood glucose level in the blood with high efficiency.

### BRIEF DESCRIPTION OF DRAWINGS

(A) and (B) of FIG. 1 are graphs showing measurement results of the total cholesterol concentration in the blood and the neutral fat concentration in the blood, respectively, in Example 4, Comparative Example 4, and Reference Example.

(A) and (B) of FIG. 2 are graphs showing measurement results of the glucose level in the blood and the cholesterol concentration in the liver after the completion of a test, respectively, in Example 4, Comparative Example 4, and Reference Example.

FIG. 3 is a graph showing measurement results of the relative liver weight to the weight of a model rat in Example 4, Comparative Example 4, and Reference Example.

FIG. 4 is a graph showing measurement results of the selective adsorption rate of sodium cholate in Example 5, Comparative Example 5A, and Comparative Example 5B.

FIG. 5 is a graph showing measurement results of the pore size distribution calculated by the Non Localized Density Functional Theory in Example 1B, Comparative Example 2, and Comparative Example 5B.

### MODE(S) FOR CARRYING OUT THE INVENTION

Hereinafter, although the present invention will be described based on Examples with reference to the drawings, the present invention is not limited to Examples, and various numerical values or materials in Examples are given for exemplary purposes. It should be noted that the description will be made in the following order.

1. General description of cholesterol lowering agent, neutral fat lowering agent, blood glucose level lowering agent, cholesterol adsorbent, neutral fat adsorbent, healthy food, health supplement, food with nutrient function claims, food for specified health use, quasi-drug, and pharmaceutical drug according to present invention
2. Example 1 (cholesterol lowering agent and cholesterol adsorbent according to present invention)
3. Example 2 (neutral fat lowering agent and neutral fat adsorbent according to present invention)
4. Example 3 (modification of Example 1)
5. Example 4 (cholesterol lowering agent, neutral fat lowering agent, and blood glucose level lowering agent according to present invention)
6. Example 5 (adsorbents according to first embodiment to third embodiment of present invention)
7. Example 6 (healthy food, health supplement, food with nutrient function claims, food for specified health use, quasi-drug, and pharmaceutical drug according to present invention), and others

### General Description of Cholesterol Lowering Agent, Neutral Fat Lowering Agent, Blood Glucose Level Lowering Agent, Cholesterol Adsorbent, Neutral Fat Adsorbent, Adsorbent, Healthy Food, Health Supplement, Food with Nutrient Function Claims, Food for Specified Health Use, Quasi-Drug, and Pharmaceutical Drug According to Present Invention

As a raw material of the porous carbon material in the present invention, a plant-based material can be used. Here, examples of the plant-based material include, but not limited to, chaff or straw of rice (rice plant), barley, wheat, rye, Japanese millet, foxtail millet, or the like, coffee beans, tea leaves (e.g., leaves of green tea, black tea, or the like), sugar canes (more specifically, bagasse), corns (more specifically, corn cores), rinds of fruits (e.g., rinds of orange or banana), reeds, and Wakame seaweed stem. In addition thereto, for example, vascular plants growing on land, pteridophytes, bryophytes, algae, and marine plants may be used as the plant-based material. It should be noted that one of these materials may be used alone as a raw material, or two or more of them may be used in combination. Moreover, the shape and form of the plant-based material are not particularly limited, and the plant-based material may be, for example, chaff or straw as it is, or a dried product. Furthermore, those subject to various treatments such as a fermentation treatment, a roasting treatment, and an extraction treatment in food and beverage processes of beer, liquor, or the like may be used. In particular, it is favorable to use the straw or chaff obtained after a process of threshing or the like from the viewpoint of recycling of industrial wastes. These processed straw and chaff can be easily obtained in large amounts from, for example, an agricultural cooperative, an alcoholic beverage maker, a food company, and a food processing company.

In the case where the raw material of the porous carbon material in the present invention is a plant-based material containing silicon (Si), specifically, a plant-based material having a silicon (Si) content of 5% by weight or more is used as the raw material of the porous carbon material, but the raw material is not limited thereto. The porous carbon material has desirably a silicon (Si) content of 5% by weight or less, favorably, 3% by weight or less, more favorably, 1% by weight or less.

The porous carbon material in the present invention can be obtained by, for example, carbonizing a plant-based material at 400° C. to 1,400° C. and treating it with an acid or alkali. In such a method of producing the porous carbon material in the present invention (hereinafter referred to as simply “method of producing the porous carbon material” in some cases), a material obtained by carbonizing a plant-based material at 400° C. to 1,400° C., which is not yet treated with an acid or alkali is referred to as “porous carbon material precursor” or “carbonaceous substance.”

In the method of producing the porous carbon material, after the treatment with an acid or alkali, a step of applying an activation process may be included, or after the application of an activation process, the treatment with an acid or alkali may be performed. Moreover, in the method of producing the porous carbon material including such a favorable form, before the carbonization of the plant-based material, the plant-based material may be subject to a heat treatment (pre-carbonization treatment) at the temperature lower than that for carbonizing the plant-based material (e.g., 400° C. to 700° C.) under the condition of oxygen insulation depending on the plant-based material to be used. In this way, a tar component, which will be produced in the process of carbonization, can be extracted, resulting in decrease or removal of the tar component, which will be produced in the process of carbonization. It should be noted that the condition of oxygen insulation can be obtained by creating an atmosphere with an inert gas such as a nitrogen gas and an argon gas, by creating a vacuum atmosphere, or by steaming and baking the plant-based material in a way. Moreover, in the method of producing the porous carbon material, in order to decrease a mineral component or water contained in the plant-based material or to prevent the generation of odor in the process of the carbonization, the plant-based material may be immersed in alcohol (e.g., methyl alcohol, ethyl alcohol, and isopropyl alcohol) depending on the plant-based material to be used. It should be noted that in the method of producing the porous carbon material, the pre-carbonization treatment may be performed thereafter. Examples of the material that is favorably subject to the heat treatment in an inert gas include a plant that generates a large amount of pyroligneous acid (tar and light crude oil component). Moreover, examples of the material that is favorably subject to the pre-treatment with alcohol include algae containing a large amount of iodine or various minerals.

In the method of producing the porous carbon material, the plant-based material is carbonized at 400° C. to 1,400° C. Here, the carbonization generally means a conversion of an organic material (the plant-based material in the case of the porous carbon material in the present invention) into a carbonaceous substance by a heat treatment (see, for example, JIS M0104-1984). It should be noted that examples of the atmosphere for the carbonization include an atmosphere of oxygen insulation, specifically, a vacuum atmosphere, an atmosphere with an inert gas such as a nitrogen gas and an argon gas, and an atmosphere in which the plant-based material is steamed and baked in a way. Examples of the rate of temperature increase until the carbonization temperature include, but not limited to, 1° C./minute or higher, favorably 3° C./minute or higher, more favorably 5° C./minute or higher under such an atmosphere. Moreover, examples of the upper limit of the carbonization time period include, but not limited to, 10 hours, favorably, 7 hours, more favorably 5 hours. The lower limit of the carbonization time period may be a time period when the plant-based material can be surely carbonized. Moreover, the plant-based material may be pulverized to particles having a desired particle size or subject to a classification as necessary. The plant-based material may be previously washed. Alternatively, the obtained porous carbon material precursor or the porous carbon material may be pulverized to particles having a desired particle size or subject to a classification as necessary. Alternatively, the porous carbon material after the activation treatment may be pulverized to particles having a desired particle size or subject to a classification as necessary. Furthermore, the finally obtained porous carbon material may be subject to a sterilization treatment. The form, configuration, or structure of the furnace used for the carbonization is not limited, and a continuous furnace or batch furnace may be used.

In the method of producing the porous carbon material, as described above, if the activation treatment is performed, it is possible to increase the number of micropores having a pore size smaller than 2 nm (which will be described later). Examples of the activation treatment include a gas activation method and a chemical activation method. Here, the gas activation method is a method in which oxygen, water vapor, a carbonic acid gas, air, or the like is used as an activator, and the porous carbon material is heated under such a gas atmosphere at 700° C. to 1,400° C., favorably 700° C. to 1,000° C., more favorably 800° C. to 950° C. for several tens of minutes to several hours, here developing the microstructure by a volatile component or a carbon molecule in the porous carbon material. It should be noted that more specifically, the heating temperature may be appropriately selected, based on the kind of the plant-based material, the kind and concentration of the gas, or the like. The chemical activation method is a method in which the activation is performed using zinc chloride, iron chloride, calcium phosphate, calcium hydroxide, magnesium carbonate, potassium carbonate, sulfuric acid, or the like instead, of oxygen or water vapor used in the gas activation method, the resulting product is washed with hydrochloric acid, its pH is adjusted with an alkaline aqueous solution, and it is dried.

As described above, the surface of the porous carbon material in the present invention may be subject to a chemical treatment or molecular modification. Examples of the chemical treatment include a treatment in which a carboxy group is produced on the surface by a nitric acid treatment. Moreover, by performing a treatment similar to the activation treatment using water vapor, oxygen, alkali, or the like, various functional groups such as a hydroxyl group, a carboxy group, a ketone group, and an ester group can be produced on the surface of the porous carbon material. Furthermore, the molecular modification can be performed also by a chemical reaction with chemical species or protein having groups capable of reacting with the porous carbon material, such as a hydroxyl group, a carboxy group, and an amino group.

In the method of producing the porous carbon material, a silicon component in the plant-based material after the carbonization is removed by the treatment with an acid or alkali. Here, examples of the silicon component include silicon oxides such as silicon dioxide, silicon oxide, and silicon oxide salt. As described above, by removing the silicon component in the plant-based material after the carbonization, it is possible to obtain the porous carbon material having a high specific surface area. In some cases, the silicon component in the plant-based material after the carbonization may be removed based on a dry etching method.

The porous carbon material in the present invention may contain non-metal elements such as magnesium (Mg), potassium (K), calcium (Ca), phosphorous (P), and sulphur (S), or metal elements such as transition elements. Examples of the magnesium (Mg) content include 0.01% by weight or more and 3% by weight or less, examples of the potassium (K) content include 0.01% by weight or more and 3% by weight or less, examples of the calcium (Ca) content include 0.05% by weight or more and 3% by weight or less, examples of the phosphorous (P) content include 0.01% by weight or more and 3% by weight or less, and examples of the sulphur (S) content include 0.01% by weight or more and 3% by weight or less. It should be noted that the contents of these elements are favorably low from the viewpoint of increase in the specific surface area value. It is needless to say that the porous carbon material may contain elements other than those described above, and the range of the contents of the various elements descried above can be changed.

The various elements in the porous carbon material in the present invention can be analyzed by using, for example, an energy dispersive X-ray analyzer (e.g., JED-2200F manufactured by JEOL Ltd.) according to energy dispersive spectrometry (EDS). Here, the measurement conditions may include a scanning voltage of 15 kV and an illumination current of 10 μA.

The porous carbon material in the present invention has a lot of pores. The pores include “mesopores” having a pore size of 2 nm to 50 nm, and “micropores” having a pore size smaller than 2 nm. Specifically, the porous carbon material in the present invention includes, for example, a lot of pores having a pore size of 20 nm or less, particularly, a lot of pores having a pore size of 10 nm or less, as the mesopores. Moreover, it includes, for example, a lot of pores having a pore size of about 1.9 nm, pores having a pore size of about 1.5 nm, and pores having a pore size of about 0.8 nm to 1 nm, as the micropores. The porous carbon material in the present invention has a pore volume of 0.1 cm³/g or more measured by the BJH method and the MP method. However, more favorably, the pore volume is 0.3 cm³/g or more.

The porous carbon material in the present invention favorably has a specific surface area value of 50 m²/g or more, more favorably 100 m²/g or more, further more favorably 400 m²/g or more, which is measured by the nitrogen BET method (hereinafter referred to as simply “specific surface area value” in some cases), for obtaining more excellent functionality.

The nitrogen BET method is a method in which an adsorption isotherm is measured by adsorbing and desorbing nitrogen to/from an adsorbent (here, the porous carbon material) as an admolecule, and the measured data are analyzed based on a BET formula represented by a formula (1). The specific surface are the pore volume, or the like can be calculated based on this method. Specifically, in the case where the specific surface area value is calculated by the nitrogen BET method, first, an adsorption isotherm is obtained by adsorbing and desorbing nitrogen to/from the porous carbon material as an admolecule. Then, [p/{Vₐ(p₀−p)}] is calculated based on the formula (1) or a formula (1′) obtained by modifying the formula (1), from the obtained adsorption isotherm, and is plotted to the equilibrium relative pressure(p/p₀). Then, a slope s (=[(C−1)/(C·Vₘ)]) and an intercept i (=[1/(C·Vₘ)]) are calculated based on a least squares method, regarding the plot as a straight line. Then, Vₘ and C are calculated based on a formula (2-1) and a formula (2-2) from the obtained slope s and the intercept i. Furthermore, a specific surface area a_{sBET} is calculated based on a formula (3) from Vₘ (see a manual for BELSORP-mini and BELSORP analysis software manufactured by BEL JAPAN INC., pages 62 to 66). It should be noted that the nitrogen BET method is a measurement method in accordance with JIS R 1626-1996 “Measuring Methods for the Specific Surface Area of Fine Ceramic Powders by Gas Adsorption Using the BET Method.”

Vₐ=(Vₘ·C·p)/[(p₀−p){1+(C−1)(p/p₀)}] (1)

[p/{Vₐ(p₀−p)}]=[(C−1)/(C·Vₘ)](p/p₀)+[1/(C·Vₘ)] (1′)

Vₘ=1/(s+i) (2-1)

C=(s/i)+1 (2-2)

a_{sBET}=(Vₘ·L·σ)/22414 (3)

In the formulae described above

Vₐ: adsorbed amount
Vₘ: adsorbed amount of monolayer
p: nitrogen pressure in equilibrium
p₀: saturated vapor pressure of nitrogen
L: Avogadro number
σ: cross-sectional area of adsorbed nitrogen

In the case where the pore volume Vₚ is calculated by the nitrogen BET method, for example, the adsorption data of the obtained adsorption isotherm are linearly interpolated, and an adsorbed amount V is obtained at relative pressure, which is set as relative pressure in a pore volume calculation. The pore volume Vₚ can be calculated based on a formula (4) from the adsorbed amount V (see a manual for BELSORP-mini and BELSORP analysis software manufactured by BEL JAPAN INC., pages 62 to 65). It should be noted that the pore volume based on the nitrogen BET method may be referred to as simply “pore volume” in some cases.

Vₚ=(V/22414)×(M_{g}/ρ_{g}) (4)

In the formula (4),

V: adsorbed amount at relative pressure
M_{g}: molecular weight of nitrogen
ρ_{g}: density of nitrogen

The pore size of the mesopore can be calculated as a distribution of pores based on, for example, the BJH method from a rate of change in pore volume with respect to the pore size. The BJH method is a method widely used as a pore distribution analysis method. In the case where the pore distribution analysis is performed based on the BJH method, first, an adsorption isotherm is obtained by adsorbing and desorbing nitrogen to/from the porous carbon material as an admolecule. Then, the thickness of an adsorption layer and an inner diameter (twice a core radius) of a pore generated when admolecules (e.g., nitrogen) are gradually desorbed from a state in which the pore is filled with the admolecules are obtained based on the obtained adsorption isotherm. A pore radius rₚ is calculated based on a formula (5), and a pore volume calculated cased on a formula (6). Then, by plotting a rate of change in pore volume (dVₚ/drₚ) with respect to a pore size (2rₚ) from the pore radius and the pore volume, a pore distribution curve is obtained (see a manual for BELSORP-mini and BELSORP analysis software manufactured by BEL JAPAN INC., pages 85 to 88).

rₚ=t+rₖ (5)

Vₚₙ=Rₙ·dVₙ−Rₙ·dtₙ·c·ΣAₚⱼ (6)

Where

Rₙ=rₚₙ²/(rₖₙ−1+dtₙ)² (7)

In the formulae described above,

rₚ: pore radius
rₖ: core radius (inner diameter/2) in the case where an adsorption layer having a thickness t adsorbs to the inner wall of a pore having a pore radius rₚ under the pressure
Vₚₙ: pore volume when the desorption of nitrogen occurs at the n-th time
dVₙ: change amount at that time
dtₙ: change amount in the thickness tₙ of the adsorption layer when the desorption of nitrogen occurs at the n-th time
rₖₙ: core radius at that time
c: fixed value
rₚₙ: pore radius when the desorption of nitrogen occurs at the n-th time
Moreover, ΣAₚⱼ represents an integration value of the area of the wall surface of the pore from j=1 to j=n−1

The pore size of the micropore can be calculated based on, for example, the MP method from a rate of change in pore volume with respect to the pore size as a pore distribution. In the case where the pore distribution analysis is performed by the MP method, first, an adsorption isotherm is obtained by adsorbing nitrogen to the porous carbon material. Then, the adsorption isotherm is converted into pore volumes with respect to a thickness t of the adsorption layer (t-plotted). Then, it is possible to obtain the pore distribution curve based on curvatures of the plots (change amount of pore volume with respect to the change amount of the thickness t of the adsorption layer) (see a manual for BELSORP-mini and BELSORP analysis software manufactured by BEL JAPAN INC., pages 72 to 73, and 82).

Alternatively, the porous carbon material may have a configuration in which a peak appears in a range of 1×10⁻⁷ m to 5×10⁻⁶ m in the pore distribution obtained by the mercury porosimetry, and a peak appears in a range of 2 nm to 20 nm in the pore distribution obtained by the BJH method. Then, in this case, furthermore, the porous carbon material favorably has a configuration in which a peak appears in a range of 2×10⁻⁷ m to 2×10⁻⁶ m in the pore distribution obtained by the mercury porosimetry, and a peak appears in a range of 2 nm to 10 nm in the pore distribution obtained by the BJH method. The measurement of the pores by the mercury porosimetry conforms to JIS R1655:2003 “Test Methods for Pore Size Distribution of Fine Ceramic Green Body by Mercury Porosimetry.”

The cholesterol lowering agent, neutral fat lowering agent, blood glucose level lowering agent, cholesterol adsorbent, neutral fat adsorbent, healthy food, health supplement, food with nutrient function claims, food for specified health use, quasi-drug, or pharmaceutical drug according to the present invention may take a form in which the porous carbon material included in them has the specific surface area value of 10 m²/g or more, which is measured by the nitrogen BET method, and the total volume of pores having a diameter of 1×10⁻⁹ m to 1×10⁻⁷ m of 0.1 cm³/g or more, which is obtained by the Non Localized Density Functional Theory (NLDFT). Alternatively, it is possible to take a form in which the porous carbon material has the specific surface area value of 10 m²/g or more, which is measured by the nitrogen BET method, at least one peak appears in the range of 3 nm to 20 nm in the pore size distribution obtained by the Non Localized Density Functional Theory, and the proportion of the total volumes of pores having a pore size in the range of 3 nm to 20 nm is 0.2 or more of the entire pore volumes.

In the Non Localized Density Functional Theory (NLDFT) prescribed in JIS Z8831-2:2010 “Pore Size Distribution and Porosity of Powders (Solid Materials)—Part 2: Analysis of Mesopores and Macropores by Gas Adsorption,” and JIS Z88313:2010 “Pore Size Distribution and Porosity of Powders (Solid Materials)—Part 3: Analysis of Micropores by Gas Adsorption,” software attached to an automatic specific surface area/pore distribution measuring apparatus, “BELSORP-MAX,” manufactured by Bel Japan Inc., is used as analysis software. A model is formed so as to have a cylindrical shape and carbon black (CB) is assumed as the prerequisite, a distribution function of a pore distribution parameter is set as “no-assumption,” and the obtained distribution data are subject to smoothing.

The porous carbon material precursor is treated with an acid or alkali. Specific examples of the treatment method include a method in which the porous carbon material precursor is immersed in an acid or alkaline aqueous solution, and a method in which the porous carbon material precursor is reacted with an acid or alkali in a gas phase. More specifically, in the case where the porous carbon material precursor is treated with an acid, examples of the acid include acidic fluorine compounds such as hydrogen fluoride, hydrofluoric acid, ammonium fluoride, calcium fluoride, and sodium fluoride. In the case where the fluorine compound is used, the amount of fluorine atoms may only need to be 4 times the amount of the silicon atoms in the silicon component contained in the porous carbon material precursor, and the concentration of the fluorine compound aqueous solution is favorably 10% by weight or more. In the case where the silicon component (e.g., silicon dioxide) contained in the porous carbon material precursor is removed by hydrofluoric acid, the silicon dioxide is reacted with the hydrofluoric acid as shown in a chemical formula (A) or chemical formula (B), and is removed as hexafluorosilicic acid (H₂SiF₆) or silicon tetrafluoride (SiF₄), thereby obtaining the porous carbon material. Then, it only needs to wash and dry the porous carbon material thereafter.

SiO₂+6HF→H₂SiF₆+2H₂O (A)

SiO₂+4HF→SiF₄+2H₂O (B)

Moreover, in the case where the porous carbon material precursor is treated with an alkali (base), examples of the alkali include sodium hydroxide. In the case where the alkali aqueous solution is used, the pH of the aqueous solution only needs to be 11 or more. In the case where the silicon component (e.g, silicon dioxide) contained in the porous carbon material precursor is removed by a sodium hydroxide aqueous solution, the silicon dioxide is reacted as shown in a chemical formula (C) by heating the sodium hydroxide aqueous solution, and is removed as sodium silicate (Na₂SiO₃), thereby obtaining the porous carbon material. Moreover, in the case where the treatment is performed by reacting sodium hydroxide in a gas phase, sodium hydroxide in the solid state is reacted as shown in the chemical formula (C) by heating the sodium hydroxide, and is removed as sodium silicate (Na₂SiO₃), thereby obtaining the porous carbon material. Then, it only needs to wash and dry the porous carbon material.

SiO₂+2NaOH→Na₂SiO₃+H₂O (C)

Alternatively, as the porous carbon material in the present invention, for example, a porous carbon material having holes with a three-dimensional regularity (porous carbon material having a so-called inverse opal structure), which is disclosed in Japanese Patent Application Laid-open No. 2010-106007, specifically, a porous carbon material having spherical holes three-dimensionally arranged with a mean diameter of 1×10⁻⁹ m to 1×10⁻⁵ m and a surface area of 3×10² m²/g or more, favorably, a porous carbon material having holes macroscopically arranged in an arrangement state corresponding to a crystalline structure or holes microscopically arranged in an arrangement state corresponding to (111) plane orientation in a face-centered cubic structure on its surface may be used.

### Example 1

Example 1 relates to the cholesterol lowering agent and cholesterol adsorbent according to the present invention (hereinafter collectively referred to as simply cholesterol lowering agent in Example 1). The cholesterol lowering agent in Example 1 includes a porous carbon material having a specific surface area value of 10 m²/g or more measured by the nitrogen BET method, and a pore volume of 0.1 cm³/g or more measure by the BJH method and the MP method. It should be noted that the raw material of the porous carbon material is a plant-based material containing silicon. Then, pores (mesopores) by the BJH method and pores (micropores) by the MP method are obtained by, at least, removing silicon from the plant-based material containing silicon.

Moreover, the porous carbon material in Example 1 includes a porous carbon material having a specific surface area of 10 m²/g or more measure by the nitrogen BET method and the total volume of pores having a diameter of 1×10⁻⁹ m to 1×10⁻⁷ m of 0.1 cm³/g or more (favorably 0.2 cm³/g or more), which is obtained by the Non Localized Density Functional Theory. Furthermore, the porous carbon material in Example 1 includes a porous carbon material having a specific surface area value of 10 m²/g or more, which is measured by the nitrogen BET method, and in which at least one peak appears in the range of 3 nm to 20 nm in the pore size distribution obtained by the Non Localized Density Functional Theory and the proportion of the total volumes of pores having a pore size in the range of 3 nm to 20 nm is 0.2 or more of the entire pore volumes (specifically, the proportion is 0.479 and the entire pore volume is 1.33 cm³/g).

In Example 1, rice (rice plant) chaff was used as the plant-based material being the raw material of the porous carbon material. Then, the porous carbon material in Example 1 is obtained by carbonizing the chaff serving as the raw material to convert the chaff into the carbonaceous substance (porous carbon material precursor), and then applying an acid treatment thereto. Hereinafter, a method of producing the porous carbon material in Example 1 will be described.

In the method of producing the porous carbon material in Example 1, the plant-based material was carbonized at 400° C. to 1,400° C., and then the resulting product was treated with an acid or alkali. Thus, the porous carbon material was obtained. That is, first, pulverized chaff (chaff of Isehikari produced in Kagoshima Prefecture having a silicon (Si) content of 10% by weight) is subject to a heat treatment (pre-carbonation treatment) in an inert gas. Specifically, the chaff was heated in a flow of nitrogen a 500° C. for 5 hours to be carbonized, thereby obtaining carbide. It should be noted that by performing such a treatment, it is possible to decrease or remove a tar component, which will be produced in the next carbonization. After that, 10 g of the carbide was put in an alumina crucible, and the temperature therein was raised 800° C. at a rate of temperature increase of 5° C./minute in a flow of nitrogen (10 liters/minute). Then, the carbonization was performed at 800° C. for 1 hour, and the carbide was converted into a carbonaceous substance (porous carbon material precursor). After that, the carbonaceous material was cooled to room temperature. It should be noted that a nitrogen gas was kept flowing during the carbonization and the cooling. Next, the porous carbon material precursor was subject to an acid treatment by being immersed in 46% by volume of a hydrofluoric acid aqueous solution for one night, and then was washed with water and ethyl alcohol until its pH reached 7. Subsequently, it was dried at 120° C., and then was subject to an activation treatment by being heated at 900° C. in a flow of water vapor for 3 hours, thereby obtaining the porous carbon material in Example 1. It should be noted that in the case where the porous carbon material is subject to a chemical treatment or molecular modification, specifically, for example, in the case where an amino group is produced on the surface of the porous carbon material, 0.50 g of the obtained porous carbon material is added to 150 ml of a saturated urea aqueous solution, and the resulting product was agitated at room temperature for 24 hours. The product obtained by filtering the suspension only needs to be cacined at 450° C. in a nitrogen atmosphere. It should be noted that the porous carbon material thus obtained is referred to as “porous carbon material in Example 1A.” Moreover, a porous carbon material obtained by performing the calcination or the like without the treatment with the saturated urea aqueous solution, i.e., a porous carbon material obtained through the same step as that in the porous carbon material in Example 1A except that the treatment with the saturated urea aqueous solution is not performed is referred to as “porous carbon material in Example 1B.”

Low molecular weight sodium alginate (having molecular weight of 50,000 to 100,000) manufactured by Wako Pure Chemical Industries, Ltd. was used for a test as Comparative Example 1A. Furthermore, a chitosan powder manufactured by Sigma-Aldrich Co. LLC. was used for a test as Comparative Example 1B. It should be noted that the molecular weight of sodium alginate is normally 200,000 to 2,000,000.

The nitrogen adsorption and desorption test was performed using BELSORP-mini (manufactured by Bel Japan Inc.) as a measuring device for obtaining the specific surface area and pore volume. As the measuring condition, the equilibrium relative pressure (p/p₀) was set to 0.01 to 0.99. Then, based on the BELSORP analysis software, the specific surface area and pore volume were calculated. Moreover, the nitrogen adsorption and desorption test was performed using the measuring device described above, and the pore size distributions of the mesopores and micropores were calculated based on the BJH method and the MP method by using the BELSORP analysis software. Moreover, the pore of the porous carbon material was measured by the mercury porosimetry. Specifically, the measurement according to the mercury porosimetry was performed by using a mercury porosimeter (PASCAL440 manufactured by Thermo Electron Corporation). The area for measuring the pore was set to 10 μm to 2 nm. Furthermore, in the measurement based on the Non Localized Density Functional Theory (NLDFT), an automatic specific surface area/pore distribution measuring apparatus, “BELSORP-MAX,” manufactured by Bel Japan Inc. was used under the following conditions.

Prerequisite for analysis: none
Prerequisite for pore cylindrical shape
Number of smoothing treatments: 10 times
It should be noted that when the measurement was performed, the sample was dried at 200° C. for 3 hours as the pre-treatment thereof.

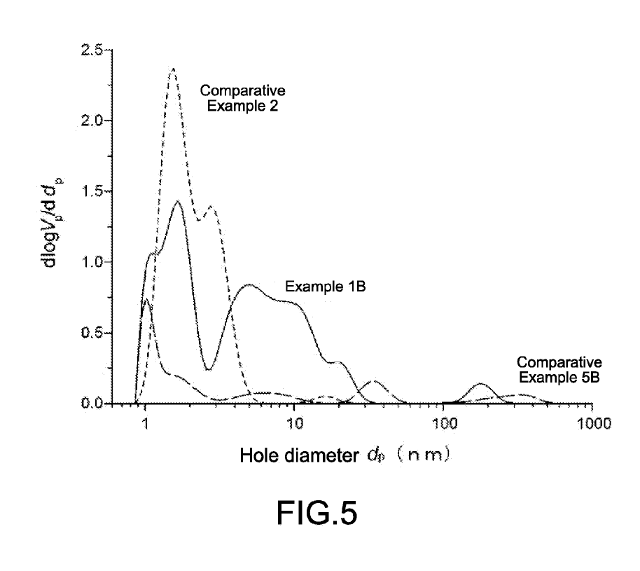

The specific surface area and pore volume in Example 1B were measured, and the results shown in Table 1 were obtained. It should be noted that in Table 1, “specific surface area” and “entire pore volume” indicate the values of the specific surface area and the entire pore volume measured by the nitrogen BET method, and units thereof are m²/g and cm³/g, respectively. Moreover, “MP method,” “BJH method,” and “mercury porosimetry” indicate measurement results of pore volume (micropores) measured by the MP method, measurement results of pore volume (mesopores) measured by the BJH method, and measurements results of the entire pore volume measured by the mercury porosimetry, respectively, and units thereof are cm³/g. It should be noted that the measurement results of the specific surface area and pore volume in Example 1A are almost the same as those in Example 1B. Moreover, FIG. 5 shows a graph of measurement results of the pore size distributions in Example 1B, Comparative Example 2, and Comparative Example 5B, which are obtained by the Non Localized Density Functional Theory.

TABLE 1

| | Specific | | | | |
|---|---|---|---|---|---|
| | surface | Entire pore | MP | BJH | Mercury |
| | area | volume | method | method | porosimetry |
| Example 1B | 1290 | 0.87 | 0.44 | 0.70 | 2.7 |
| Comparative | 1231 | 0.57 | 0.56 | 0.04 | 1.7 |
| Example 2 | | | | | |
| Comparative | 1559 | 0.76 | 0.75 | 0.14 | 1.5 |
| Example 3B | | | | | |
| Comparative | 1095 | 0.82 | 0.49 | 0.45 | 2.1 |
| Example 5A | | | | | |
| Comparative | 362 | 0.20 | 0.16 | 0.07 | <1.5 |
| Example 5B | | | | | |

The adsorbed amount of cholesterol (bile acids) in a simulated intestinal fluid was measured by the following method.

Bile acids and fatty acids were added to saline (aqueous solution obtained by dissolving 9 g of sodium chloride in 1,000 ml of water) so as to have a concentration of 6.4 mmol and 9.9 mmol, respectively, and were agitated to be dissolved. Thus, the simulated intestinal fluid (test solution) was prepared.

In a vial container, 10.0 ml of the test solution was put, and 10.0 mg of the cholesterol lowering agents in Example 1A and Example 1B, and the samples in Comparative Example 1A and Comparative Example 1B were added thereto to be shaken in a thermostatic bath at 37° C. for 1 hour. Next, the suspension was filtered through a filter of 0.45 μm. Thus, a subject solution was obtained. Then, the absorbance of the filtrate was measured (measurement wavelength of 234 nm), and the adsorbed amount of bile acids (cholesterol) was calculated.

Examples of the bile acids include cholic acid, deoxycholic acid, lithocholic acid, chenodeoxycholic acid, sodium salts or potassium salts of these substances, and conjugated bile acid in which these substances are bonded to glycine, taurine, or the like in an amide form. One of these bile acids may be used alone, or two or more of these bile acids may be used in combination. It should be noted that examples of the conjugated bile acid include glycocholic acid, taurocholic acid, glycochenodeoxycholic acid, taurodeoxycholic acid, and sodium salts or potassium salts of these substances. One of the conjugated bile acids may be used alone, or two or more of the conjugated bile acids may be used in combination. Moreover, the bile acids may be bile acid as it is, or may be bile acid mixed micelle formed of bile acid and a compound capable of forming a micelle with bile acids. Examples of the compound capable of forming a micelle with bile acids include fatty acids such as oleic acid, palmitic acid, stearic acid, and linolenic acid, and salts thereof.

Moreover, examples of the fatty acids include fatty acid contained in a normal intestinal fluid in human beings, e.g., at least one kind of compound selected from a group consisting of sodium oleate, linoleic acid sodium salt, linolenic acid sodium salt, sodium ricinoleate, sodium caproate, sodium caprylate, sodium caprate, sodium laurate, sodium myristate, sodium palmitate, and sodium stearate.

In Example 1, as the bile acid, specifically, sodium cholate was used. On the other hand, as the fatty acid, specifically, sodium oleate was used. In the tests, the adsorbed amount per 1 g of the cholesterol lowering agents in Examples 1A and Example 1B, and the samples in Comparative Example 1A and Comparative Example 1B was calculated based on the following formula. The results are shown in the following Table 2. It can be understood that the adsorption capacity of cholesterol in Example 1A and Example 1B is superior to that in Comparative Example 1A and Comparative Example 1B.

Adsorbed amount of bile acids(cholesterol)(mg/g)=(molecular weight of bile acids)×{(mol concentration of solution before adsorption)−(mol concentration of solution after adsorption)}/{weight(g)of lowering agent,adsorbent,or sample per 1000 ml of solution}

TABLE 2

| | Adsorbed amount of sodium cholate in |
|---|---|
| | simulated intestinal fluid (mg/g) |
| Example 1A | 872 |
| Example 1B | 695 |
| Comparative | 538 |
| Example 1A | |
| Comparative | 470 |
| Example 1B | |

### Example 2

Example 2 relates to the neutral fat lowering agent and neutral fat adsorbent according to the present invention (hereinafter collectively referred to as simply neutral fat lowering agent in Example 2). Similarly to the cholesterol lowering agent in Example 1, also the neutral fat lowering agent in Example 2 includes a porous carbon material having a specific surface area value of 10 m²/g or more, which is measured by the nitrogen BET method, and a pore volume of 0.1 cm³/g or more, which is measured by the BJH method and the MP method. In Example 2, the porous carbon material in Example 1B was used as the neutral fat lowering agent.

In Example 2, as the test solution, 300 mg/dl of a tripalmitin aqueous solution was used. Then 10.0 ml of the test solution was put in a vial container, and 10.0 mg of the samples in Example 1B and Comparative Example 2 were added thereto to be shaken in a thermostatic bath at 37° C. for 3.5 hours. It should be noted that the sample in Comparative Example 2 is one obtained by powdering commercially available activated carbon (manufactured by Wako Pure Chemical Industries, Ltd.), and the measurement results of the specific surface area and pore volume are shown in Table 1. Next, the suspension was filtered through a filter of 0.45 μm. Thus, a subject solution was obtained. Then, the amount of triglyceride in the filtrate was measured (Triglyceride E-Test Wako), and the adsorbed amount of tripalmitin (neutral fat) was obtained. The results are shown in the following Table 3. From Table 3, it can be understood that the adsorption capacity for neutral fat of the neutral fat lowering agent in Example 2 is significantly superior to that of the sample in Comparative Example 2.

TABLE 3

| | Adsorbed amount of neutral fat |
|---|---|
| | (tripalmitin) (mg/g) |
| Example 2 | 389 |
| Comparative | 55 |
| Example 2 | |

### Example 3

It has been known that negative ion exchange resin such as cholestyramine being a well-known cholesterol lowering agent has a problem that the cholesterol lowering capacity is affected by a coexistent electrolyte such as sodium chloride.

In Example 3, the porous carbon material in Example 1B was used as the cholesterol lowering agent and cholesterol adsorbent in Example 3 (hereinafter collectively referred to as simply cholesterol lowering agent in Example 3), and whether or not the cholesterol lowering capacity was affected by a coexistent electrolyte such as sodium chloride, protein, and lipid was examined. Moreover, cholestyramine was used as Comparative Example 3A, and Kremezin was used as Comparative Example 3B. The measurement results of the specific surface area and pore volume of Kremezin in Comparative Example 3B are shown in Table 1.

In Example 3, the cholesterol lowering agent in Example 3 and the samples in Comparative Example 3A and Comparative Example 3B were dried at 100° C. for 4 hours. Then, 10 mg of each of them was weighed, and was put in a vial container having a volume of 50 ml. Each of 10 ml of a solution obtained by adding 0.1% by weight of sodium chloride (NaCl) being an electrolyte to a bile acid solution (specifically including 1 mmol of a sodium cholate aqueous solution) adjusted to 1 mmol (referred to as NaCl additive), and 10 ml of a solution to which no sodium chloride is added (referred to as NaCl-free solution) was further added to the vial container, and was shaken in a thermostatic bath at 37° C. for 24 hours to be agitated. After the agitation was stopped, the cholesterol lowering agent in Example 3 and the samples in Comparative Example 3A and Comparative Example 3B were removed by being filtered through a membrane filter of 0.2 μm. Then, the concentration of free bile acid in the filtrate was measured by a measurement kit using the enzyme colorimetric method. It should be noted that it has been confirmed that the bile acid is not adsorbed to the membrane filter. The measurement results of the removal rate of the free bile acid (unit: %) is shown in the following Table 4. It was confirmed that the adsorbed amount of bile acid is affected by the addition of an electrolyte (NaCl).

TABLE 4

| | NaCl-free | NaCl |
|---|---|---|
| | solution | additive |
| Example 3 | 70 | 94 |
| Comparative | 77 | 60 |
| Example 3A | | |
| Comparative | 11 | 18 |
| Example 3B | | |

Next, the cholesterol lowering agent in Example 3, and the samples in Comparative Example 3A and Comparative Example 3B were dried at 100° C. for 4 hours. Then, 10 mg of each of them was weighed, and was put in a vial container having a volume of 50 ml. Each of 10 ml of solutions obtained by adding various amounts of sodium chloride (NaCl) being an electrolyte to the bile acid solution adjusted to 1 mmol was further added to the vial container, and was shaken in a thermostatic bath at 37° C. for 4 hours to be agitated, in the same way as described above. After the agitation was stopped, the cholesterol lowering agent in Example 3 and the samples in Comparative Example 3A and Comparative Example 3B were removed by being filtered through a membrane filter of 0.2 μm. Then, the concentration of free bile acid in the filtrate was measured by a measurement kit using the enzyme colorimetric method. The measurement results of the removal rate of the free bile acid (unit: %) is shown in the following Table 5.

TABLE 5

| | NaCl-free | 0.1 mol of | 0.2 mol of |
|---|---|---|---|
| | solution | NaCl | NaCl |
| Example 3 | 60 | 100 | 100 |
| Comparative | 100 | 33 | 29 |
| Example 3A | | | |
| Comparative | 4 | 21 | 22 |
| Example 3B | | | |

From Table 5, the adsorbed amount of cholesterol (bile acid) of cholestyramine being an existing cholesterol lowering agent (Comparative Example 3A) is significantly affected by the increase in the concentration of sodium chloride. On the other hand, the adsorbed amount of cholesterol (bile acid) of the cholesterol lowering agent in Example 3 was not decreased even under the existence of sodium chloride. Moreover, although the adsorbed amount of cholesterol (bile acid) of Kremezin used as an existing kidney disease drug (Comparative Example 3) is increased by the increase in the concentration of sodium chloride, an effect equivalent to that in Example 3 is not seen.

Next, the cholesterol lowering agent in Example 3, and the samples in Comparative Example 3A and Comparative Example 3B were dried at 100° C. for 4 hours. Then, 10 mg of each of hem was weighed, and was put in a vial container having a volume of 50 ml. Each 10 ml of solutions obtained by adding triolein (additive amount: 1 mmol) being lipid and albumin (additive amount: 1% by weight) being protein to the bile acid solution adjusted to 1 mmol with an artificial intestinal fluid was further added to vial container, and was shaken in a thermostatic bath at 37° C. for 4 hours to be agitated, in the same way as described above. After the agitation was stopped, the cholesterol lowering agent in Example 3 and the samples in Comparative Example 3A and Comparative Example 3B were removed by being filtered through a membrane filter of 0.2 μm. Then, the concentration of free bile acid in the filtrate was measured by a measurement kit using the enzyme colorimetric method. The measurement results of the removal rate of the free bile acid (unit: %) is shown in the following Table 6. It should be noted that the artificial intestinal fluid was prepared in the following way. That is, distilled water is added to 250 ml of an aqueous solution containing 0.2 mol/l of sodium hydrogenphosphate and 118 ml of an aqueous solution containing 0.2 mol/l of sodium hydroxide, and the resulting solution was prepared so as to have a total amount of 1000 ml.

TABLE 6

| | Lipid- and | | |
|---|---|---|---|
| | protein-free | Lipid | Protein |
| | solution | additive | additive |
| Example 3 | 10 | 14 | 33 |
| Comparative | 13 | 16 | 11 |
| Example 3A | | | |
| Comparative | 13 | 14 | 4 |
| Example 3B | | | |

From Table 6, the adsorbed amount of bile acid of cholestyramine being an existing cholesterol lowering agent (Comparative Example 3A) and Kremezin used as an existing kidney disease drug (Comparative Example 3) is affected to be decreased by the addition of albumin (protein). However, the adsorbed amount of cholesterol (bile acid) of the cholesterol lowering agent in Example 3 was increased by the addition of albumin (protein). Moreover, the adsorbed amount of cholesterol (bile acid) of the cholesterol lowering agent in Example 3 is not affected even by the addition of triolein (lipid).

Ad described above, it was found that the cholesterol lowering capacity of the cholesterol lowering agent in Example 3 was less affected by a coexistent electrolyte such as sodium chloride and protein, as compared to the existing cholesterol lowering agent. Moreover, it was found that the cholesterol lowering capacity of the cholesterol lowering agent in Example 3 was not affected also by coexistent lipid.

### Example 4

In Example 4, the porous carbon material in Example 1B was used as the cholesterol lowering agent, neutral fat lowering agent, and blood glucose level lowering agent (hereinafter collectively referred to as simply “lowering agent in Example 4”), and whether or not the cholesterol, neutral fat, and blood glucose level in a model rat with hyperlipidemia were decreased by the porous carbon material was evaluated.

In Example 4, the composition of the test group was as follows.

That is, in a test substance group (Example 4), a control group (Comparative Example 4), and a positive control group (Reference Example), high-cholesterol feed (powder feed containing 1% by weight of cholesterol and 0.5% by weight of cholic acid) was used as feed. Moreover, in the test substance group (Example 4), 10% by weight of the lowering agent in Example 4 was mixed in the feed as administered substance and was administered in feed. On the other hand, in the positive control group (Reference Example), gavage administration of cholestyramine was performed. Specifically, the dose of cholestyramine was set to 500 mg/kg/day, and the administration capacity was set to 10 ml/kg. Moreover, in the test substance group (Example 4), control group (Comparative Example 4), and positive control group (Reference Example), the number of animals was set to 8.

The high-cholesterol feed was prepared in the following way. That is, by using a dry powder mixer (rocking mixer RM-10-2 manufactured by Aichi Electric Co., Ltd.), cholesterol and cholic acid were mixed in powder feed so as to have a final total amount of 1% by weight and 0.5% by weight, respectively, and were prepared. Specifically, in the case where 4.0 kg of the high-cholesterol feed was produced, 400 g of cholesterol and 20 g of cholic acid were weighed. Then, the weighed cholesterol and cholic acid were mixed with a small amount of powder feed by the dry powder mixer. The operation of adding a small amount of powder feed and mixing them was repeated three to four times as a pre-treatment. Next, the remaining powder feed was put in the dry powder mixer, and the dry powder mixer was operated for 30 minutes. After that, the mixed powder feed was taken out from the dry powder mixer, and the feed for the control group and positive control group was put in a polyethylene bag to be hermetically sealed. The polyethylene bag was stored in a stainless container with a lid. The remaining high-cholesterol feed was further used for preparing the mixed feed in the test substance group (Example 4).

The mixed feed in the test substance group (Example 4) was prepared in the following way. That is, the lowering agent in Example 4 and the high-cholesterol feed were mixed by using the dry powder mixer. Specifically, 100 g of the lowering agent in Example 4 was weighed to prepare 10% by weight of the feed with respect to the prepared total amount of 1.0 kg. Then, the weight lowering agent in Example 4 and a small amount of the high-cholesterol feed were mixed by using the dry powder mixer. The operation of adding a small amount of the high-cholesterol feed and mixing them was repeated three to four times as a pre-treatment. Next, the remaining high-cholesterol feed was put in the dry powder mixer, and the dry powder mixer was operated for 30 minutes. After that, the mixed feed in the test substance group was taken out from the dry powder mixer, and was put in a polyethylene bag to be hermetically sealed. The polyethylene bag was stored in a stainless container with a lid.

As the administration solution for gavage administration of cholestyramine, the following administration solution was prepared. That is, sterile distilled water was added to 1,800 g of Questran powder (manufactured by Sanofi-aventis) (0.800 g of cholestyramine), and the resulting solution was suspended to be diluted in a measuring cylinder so as to have a total amount of 16 ml. Thus, the administration solution (50 mg/ml solution) was obtained.

The feeding method was as follows. That is, the feed was put in a powder feeder made of stainless (manufactured by Natsume Seisakusho Co., Ltd.), and was freely consumed. From the day of obtaining animals to the previous day of starting administration, powder feed CRF-1 (Oriental Yeast Co., ltd.) was supplied to all samples. After the day of starting administration, cholesterol additive feed was supplied to the control group and the positive control group, and feed obtained by mixing it with the lowering agent in Example 4 was supplied to the test substance group. The replacement of the feed was performed once a week excluding the day of autopsy (Day 29). It should be noted that the feed of all samples was collected from 8 o'clock to 9 o'clock on the day of autopsy.

The administration method was as follows. That is, in the test substance group, feed was freely consumed with the high-cholesterol feed in the powder feeder made of stainless. Moreover, in the positive control group, a polypropylene syringe barrel with a capacity of 2.5 to 5 ml and rat sonde were used, and gavage administration was performed. The administration solution was collected in the syringe barrel while being agitated by a magnetic stirrer.

The administration time period was 28 days. Cholestyramine was administered to the positive control group once a day.

The total cholesterol in the blood, neutral fat in the blood, and glucose level in the blood in each group were measured at Day 3, Day 14, and Day 29. After the completion of the test, also the cholesterol level in the liver and the relative weight of the liver to the body weight were measured.

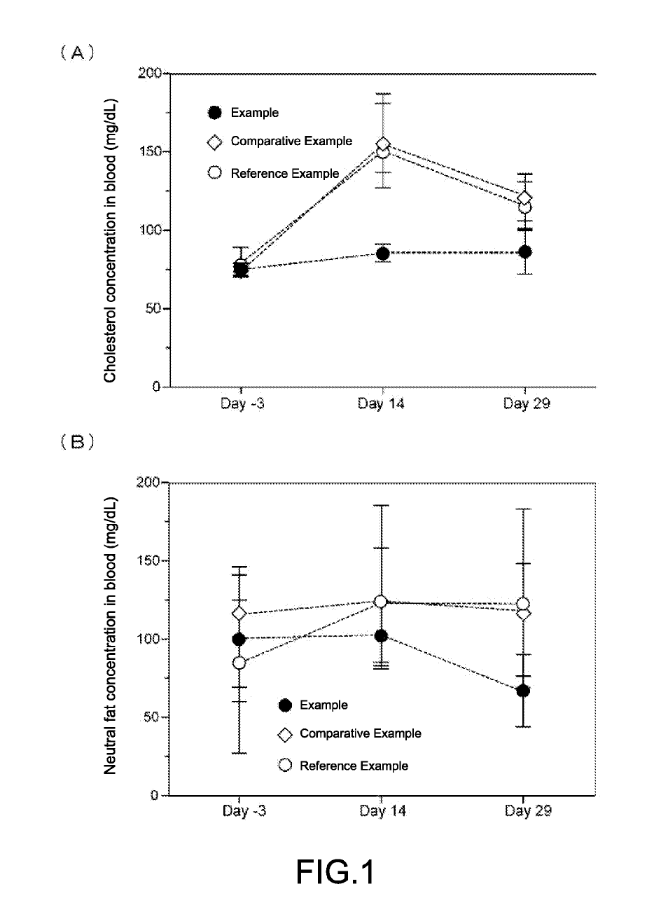

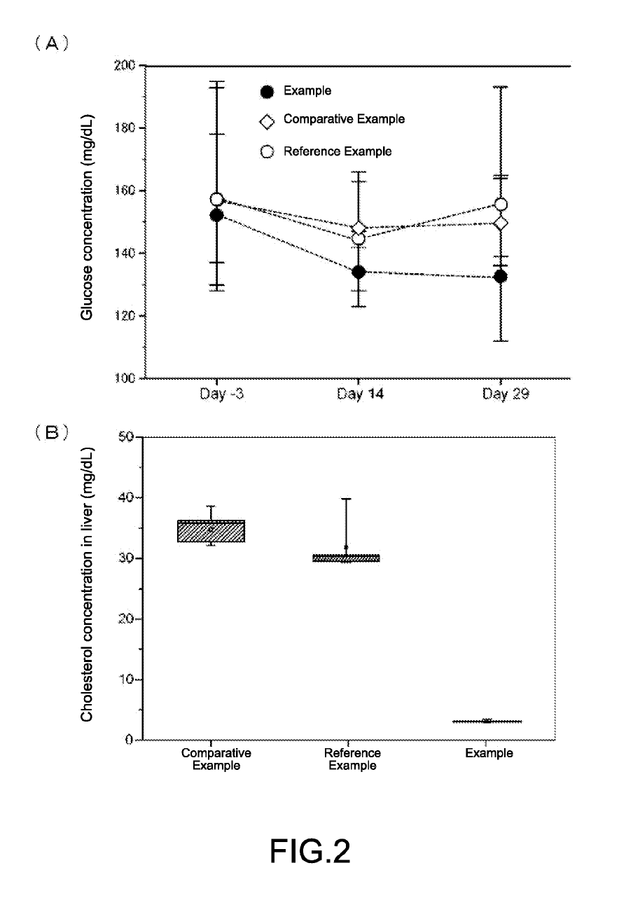

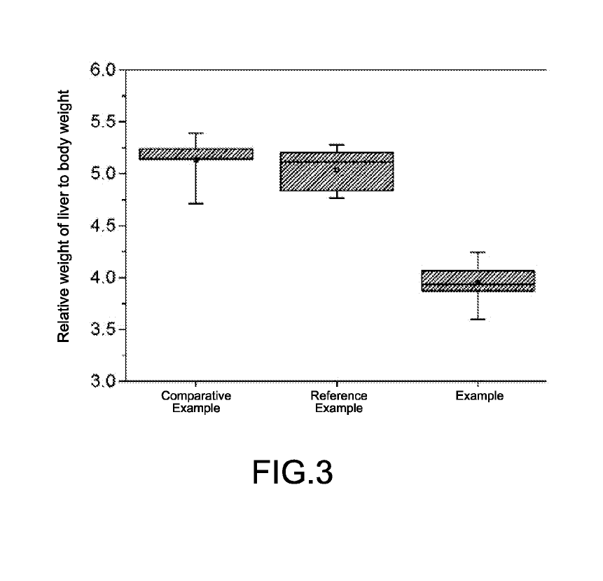

The measurement results of the total cholesterol concentration in the blood are shown in (A) of FIG. 1. The value in Example 4 was lower than those in Comparative Example 4 and Reference Example at Day 14 and Day 29, showing a significant difference. Moreover, the measurement results of the neutral fat concentration in the blood are shown in (B) of FIG. 1. The value in Example 4 was lower than those in Comparative Example 4 and Reference Example at Day 29, showing a significant difference. The measurement results of the glucose level in the blood are shown in (A) of FIG. 2. The value in Example 4 was lower than those in Comparative Example 4 and Reference Example at Day 14 and Day 29, showing a significant difference. Moreover, the measurement results of the cholesterol concentration in the liver after the completion of the test are shown in (B) of FIG. 2. The value in Example 4 was lower than those in Comparative Example 4 and Reference Example, showing a significant difference. Furthermore, the measurement results of the relative weight of the liver to the body weight are shown in FIG. 3. Because the accumulation of cholesterol was small in Example 4, the value in Example 4 was lower than those in Comparative Example 4 and Reference Example, showing a significant difference. These results indicated that the lowering agent in Example 4 had the operation of lowering the total cholesterol in the blood, neutral fat, and blood glucose level. It should be noted that in (A) and (B) of FIG. 1, (A) and (B) of FIG. 2, and FIG. 3, data in Example 4 and Comparative Example 4 have been shown as “Example” and “Comparative Example,” respectively.

### Example 5

Example 5 relates to the adsorbents including a porous carbon material according to the first embodiment to the third embodiment of the present invention.

The adsorbent in Example 5 includes a porous carbon material having an adsorbed amount of sodium cholate of 150 mg/g or more, and a selective adsorption rate of sodium cholate of 3 or more under existence of a molecule having a molecular weight of 1×10⁴ or more. Alternatively, the adsorbent in Example 5 includes a porous carbon material, includes a porous carbon material having an adsorbed amount of sodium cholate of 150 mg/g or more, and a selective adsorption rate of sodium cholate of 1.1 or more under existence of a molecule having a molecular weight of 1×10³ or less. Alternatively, the adsorbent in Example 5 includes a porous carbon material having an adsorbed amount of sodium cholate of 150 mg/g or more, and a selective adsorption rate of sodium cholate of 6 or more under existence of a mineral.

Specifically, the porous carbon material in Example 5 includes the porous carbon material in Example 1B. Moreover, the porous carbon material in Comparative Example 5A includes medicinal charcoal, and that in Comparative Example 5B includes edible charcoal. The measurement results of the specific surface area and pore volume in Comparative Example 5A and Comparative Example 5B are shown in Table 1.

The samples in Example 5, Comparative Example 5A, and Comparative Example 5B were dried, and 10 mg of them were weighed to be put in a container. On the other hand, sodium cholate was dissolved in a phosphate buffer solution, and thus 1 mmol of a sodium cholate solution was prepared. Ten ml of the sodium cholate solution was added to the container in which each sample was put. The resulting solution was shaken at 37° C. for 3 hour, and was filtered through a filter of 0.2 μm. After that, the absorbance was measured at a wavelength of 560 nm by using Total Bile Acid Test Wako (an enzyme colorimetric method). The adsorbed amount and adsorbed rate of sodium cholate were obtained based on the absorbance.

Similarly, the samples in Example 5, Comparative Example 5A, and Comparative Example 5B were dried, and 10 mg of them were accurately measured to be put in a container. On the other hand, α-amylase (molecular weight: 50,000) was dissolved in a phosphate buffer solution so as to have a concentration of 500 mg/l, and thus an α-amylase solution was prepared. Ten ml of the α-amylase solution was added to the container in which each sample was put. The resulting solution was shaken at 37° C. for 3 hour, and was filtered through a filter of 0.2 μm. After that, the absorbance was measured at a wavelength of 280 nm. The adsorbed amount and adsorbed rate of α-amylase were obtained based on the absorbance.

Similarly, the samples in Example 5, Comparative Example 5A, and Comparative Example 5B were dried, and 10 mg of them were accurately measured to be put in a container. On the other hand, vitamin K1 (molecular weight: 451) was dissolved in ethyl alcohol so as to have a concentration of 30 mg/dl, and thus vitamin K1 was prepared. Ten ml of the vitamin K1 solution was added to the container in which each sample was put. The resulting solution was shaken at 37° C. for 3 hour, and was filtered through a filter of 0.2 μm. After that, the absorbance was measured at a wavelength of 250 nm. The adsorbed amount and adsorbed rate of vitamin K1 were obtained based on the absorbance.

Similarly, the samples in Example 5, Comparative Example 5A, and Comparative Example 5B were dried, and 10 mg of them were accurately measured to be put in a container. On the other hand, calcium chloride was dissolved in a phosphate buffer solution so as to have a concentration of 10 mg/dl, and thus a calcium solution was prepared. Ten ml of the calcium solution was added to the container in which each sample was put. The resulting solution was shaken at 37° C. for 3 hour, and was filtered through a filter of 0.2 μm. After that, the absorbance was measured at a wavelength of 610 nm by using calcium E-Test Wako (MXB method). The adsorbed amount and adsorbed rate of calcium were obtained based on the absorbance.

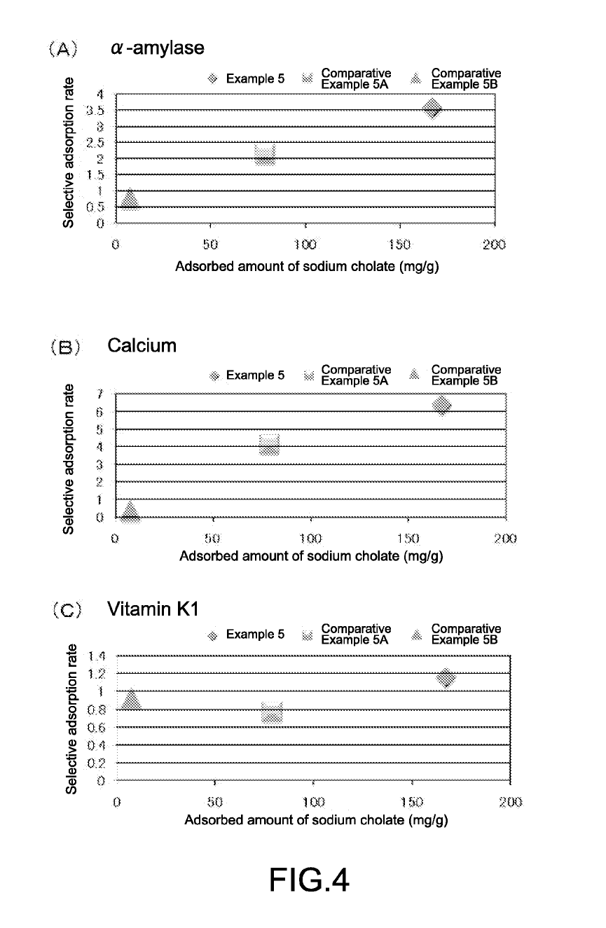

Table 7 shows the selective adsorption rate of sodium cholate to α-amylase, vitamin K1, and calcium in each sample, and Table 8 shows the adsorbed amount of sodium cholate alone (unit: mg/g). Moreover, FIG. 4 shows the measurement results of the selective adsorption rate of sodium cholate in Example 5, Comparative Example 5A, and Comparative Example 5B.

Here, the selective adsorption rate of sodium cholate is defined as, in Example 5,

(adsorbed rate of sodium cholate)/(adsorbed rate of α-amylase),
(adsorbed rate of sodium cholate)/(adsorbed rate of vitamin K1), and
(adsorbed rate of sodium cholate)/(adsorbed rate of calcium).

TABLE 7

| | | Comparative | Comparative |
|---|---|---|---|
| | Example 5 | Example 5A | Example 5B |
| α-amylase | 3.6 | 2.1 | 0.8 |
| Vitamin K1 | 1.2 | 0.8 | 0.9 |
| Calcium | 6.4 | 4.1 | 0.3 |

TABLE 8

| | Adsorbed amount of sodium |
|---|---|
| | cholate alone (mg/g) |
| Example 5 | 167.2 |
| Comparative | 78.8 |
| Example 5A | |
| Comparative | 7.4 |
| Example 5B | |

From Table 7, it can be understood that the adsorbent including a porous carbon material in Example 5 has a high selective adsorption rate of sodium cholate to α-amylase, vitamin K1, and calcium. That is, it can be understood that the adsorbent including a porous carbon material in Example 5 is superior in the adsorption capacity of sodium cholate, while it has a difficulty in adsorbing a molecule having a molecular weight of 1×10⁴ or more, a molecule having a molecular weight of 1×10³ or less, and a mineral, which are typified by α-amylase, vitamin K1, and calcium, respectively. In other words, in the case where sodium cholate and α-amylase coexist, it adsorbs sodium cholate well, while it does not adsorb α-amylase well (leaves α-amylase). In the case where sodium cholate and vitamin K1 coexist, it adsorbs sodium cholate well, while it does not adsorb vitamin K1 well (leaves vitamin K1). In the case where sodium cholate and calcium coexist, it adsorbs sodium cholate well, while it does not adsorb calcium well (leaves calcium). On the other hand, for example, the edible charcoal in Comparative Example 5B has a low selective adsorption rate of sodium cholate to α-amylase, vitamin K1, and calcium. That is, in Comparative Example 5B, it adsorbs sodium cholate, while it adsorbs most of α-amylase, vitamin K1, and calcium.

### Example 6

Example 6 relates to the healthy food, health supplement, food with nutrient function claims, food for specified health use, quasi-drug, and pharmaceutical drug according to the present invention. In Example 6, the cholesterol adsorbent described in Example 1 is included, the neutral fat adsorbent described in Example 2 is included, or the cholesterol adsorbent and neutral fat adsorbent described in Example 1 and Example 2 are included. More specifically, in Example 6, the porous carbon material in Example 1B is included.

More specifically, 95 parts by weight of sucrose fatty acid ester were added to 5 parts by weight of the cholesterol adsorbent powder in Example 3, and the resulting product was uniformly mixed to be formed into a tablet having a weight of 200 mg per a tablet by using a tableting machine. Thus, the healthy food or health supplement in Example 6 was obtained.

Moreover, the porous carbon in Example 1B in a powder form was added to drinks, and thus the food with nutrient function claims or food for specified health use in Example 6 was obtained. Alternatively, it was added to biscuit, sable, or cookie, and thus the food with nutrient function claims or food for specified health use in Example 6 was obtained.

Moreover, 98.7 parts by weight of water, 0.2 parts by weight of a fragrance, and 0.1 parts by weight of a sweetener were added to 1 part by weight of the porous carbon in Example 1B or the cholesterol adsorbent powder in Example 3, and the resulting product was agitated and dissolved, producing 100 ml of a drink per a can. Thus, the quasi-drug in Example 6 was obtained.

Furthermore, 3 parts by weight of sugar or cellulose polymer as a binder and 7 parts by weight of the porous carbon in Example 1B or the cholesterol adsorbent powder in Example 3 were mixed to be formed into a tablet. Thus, the pharmaceutical drug in Example 6 was obtained. Alternatively, the porous carbon in Example 1B or the cholesterol adsorbent powder in Example 3 was encapsulated in a capsule as it is, thereby obtaining the pharmaceutical drug in Example 6.

Although the present invention has been described based on favorable Examples, the present invention is not limited to these Examples and various modification can be made.

In the Examples, although the case where chaff is used as the raw material of the porous carbon material has been described, a different plant may be used as the raw material. Here, examples of the different material include straws, reeds or Wakame seaweed stem, vascular plants growing on land, pteridophytes, bryophytes, algae, and marine plants. One of these may be used alone, or two or more of them may be used in combination. Specifically, for example, a rice straw (e.g., Isehikari produced in Kagoshima Prefecture) is used as a plant-based material being the raw material of the porous carbon material, and the straw serving as the raw material is carbonized to be converted into a carbonaceous substance (porous carbon material precursor). Next, an acid treatment is applied to the carbonaceous substance, and thus the porous carbon material can be obtained. Alternatively, a gramineous reed is used as a plant-based material being the raw material of the porous carbon material, and the gramineous reed serving as the raw material is carbonized to be converted into a carbonaceous substance (porous carbon material precursor). Next, an acid treatment is applied to the carbonaceous substance, and thus the porous carbon material can be obtained. Moreover, in the porous carbon material obtained by being treated with an alkali (base) such as a sodium hydroxide aqueous solution instead of a hydrofluoric acid aqueous solution, similar results were obtained.

Alternatively, Wakame seaweed stem (produced in Sanriku, Iwate prefecture) is used as a plant-based material being the raw material of the porous carbon material, and the Wakame seaweed stem serving as the raw material is carbonized to be converted into a carbonaceous substance (porous carbon material precursor). Next, an acid treatment is applied to the carbonaceous substance, and thus the porous carbon material can be obtained. Specifically, first, for example, the Wakame seaweed stem is heated at about 500° C. to be carbonized. It should be noted that before the heating, for example, the Wakame seaweed stem serving as the raw material may be treated with alcohol. Specific examples of the treatment method include a method of immersion in ethyl alcohol or the like. According to this, it is possible to decrease water contained in the raw material, and to elute elements other than carbon or a mineral component contained in the porous carbon material to be finally obtained. Moreover, by the treatment with alcohol, it is possible to reduce the generation of a gas during the carbonization. More specifically, the Wakame seaweed stem is immersed in ethyl alcohol for 48 hours. It should be noted that an ultrasonic treatment is favorably applied in the ethyl alcohol. Next, the Wakame seaweed stem is heated at 500° C. for 5 hours in a flow of nitrogen to be carbonized. Thus carbide is obtained. It should be noted that by performing such a treatment (pre-carbonization treatment), it is possible to decrease or remove a tar component, which will be produced in the next carbonization. After that, 10 g of the carbide is put in an alumina crucible, and the temperature therein is raised to 1000° C. at a rate of temperature increase of 5° C./minute in a flow of nitrogen (10 liters/minute). Then, the carbonization is performed at 1.000° C. for 5 hours, and the carbide is converted into a carbonaceous substance (porous carbon material precursor). After that, the carbonaceous material is cooled to room temperature. It should be noted that a nitrogen gas is kept flowing during the carbonization and the cooling. Next, the porous carbon material precursor is subject to an acid treatment by being immersed in 46% by volume of hydrofluoric acid aqueous solution for one night, and then is washed with water and ethyl alcohol until its pH reaches 7. Then, it was finally dried, thereby obtaining the porous carbon material.

1. A cholesterol lowering agent, comprising:
a porous carbon material having a specific surface area value of 10 m²/g or more and a pore volume of 0.1 cm³/g or more, the specific surface area value measured by a nitrogen BET method, and the pore volume measured by at least one of a BJH method and an MP method.

2. The cholesterol lowering agent according to claim 1, wherein
a cholesterol lowering capacity is not affected by a coexistent protein.

3. The cholesterol lowering agent according to claim 1, wherein
a cholesterol lowering capacity is not affected by a coexistent sodium chloride.

4. A neutral fat lowering agent, comprising:
a porous carbon material having a specific surface area value of 10 m²/g or more and a pore volume of 0.1 cm³/g or more, the specific surface area value measured by a nitrogen BET method, and the pore volume measured by at least one of a BJH method and an MP method.

5. A blood glucose level lowering agent, comprising:
a porous carbon material having a specific surface area value of 10 m²/g or more and a pore volume of 0.1 cm³/g or more, the specific surface area value measured by a nitrogen BET method, and the pore volume measured by at least one of a BJH method and an MP method.

6. A cholesterol adsorbent, comprising:
a porous carbon material having a specific surface area value of 10 m²/g or more and a pore volume of 0.1 cm³/g or more, the specific surface area value measured by a nitrogen BET method, and the pore volume measured by at least one of a BJH method and an MP method.

7. The cholesterol adsorbent according to claim 6, wherein
the porous carbon material is subject to a chemical treatment or molecular modification.

8. A neutral fat adsorbent, comprising:
a porous carbon material having a specific surface area value of 10 m²/g or more and a pore volume of 0.1 cm³/g or more, the specific surface area value measured by a nitrogen BET method, and the pore volume measured by at least one of a BJH method and an MP method.

9. The neutral fat adsorbent according to claim 8, wherein
the porous carbon material is subject to a chemical treatment or molecular modification.

10. An adsorbent, comprising:
a porous carbon material having an adsorbed amount of sodium cholate of 150 mg/g or more and a selective adsorption rate of sodium cholate of 3 or more under existence of a molecule having a molecular weight of 1×10⁴ or more.

11. The adsorbent according to claim 10, wherein
the molecule having a molecular weight of 1×10⁴ or more is an enzyme or protein.

12. An adsorbent, comprising:
a porous carbon material having an adsorbed amount of sodium cholate of 150 mg/g or more and a selective adsorption rate of sodium cholate of 1.1 or more under existence of a molecule having a molecular weight of 1×10³ or less.

13. The adsorbent according to claim 12, wherein
the molecule having a molecular weight of 1×10³ or less is vitamin K.

14. An adsorbent, comprising:
a porous carbon material having an adsorbed amount of sodium cholate of 150 mg/g or more and a selective adsorption rate of sodium cholate of 6 or more under existence of a mineral.

15. The adsorbent according to claim 14, wherein
the mineral is a calcium compound or a magnesium compound.

16. A healthy food, comprising:
the cholesterol adsorbent according to claim 6.

17. A health supplement, comprising:
the cholesterol adsorbent according to claim 7.

18. A healthy food, comprising:
the neutral fat adsorbent according to claim 8.

19. A food for specified health use, comprising:
the neutral fat adsorbent according to claim 9.

20. A quasi-drug, comprising:
the neutral fat adsorbent according to claim 8.

21. A pharmaceutical drug, comprising:
the neutral fat adsorbent according to claim 9.
